(19) United States
(12) Patent Application Publication
(10) Pub. No.: US 2014/0296588 A1
(43) Pub. Date: Oct. 2, 2014

(54) PRODUCTION OF BUTADIENE AND MIXED ETHERS FROM AN OXYGENATE TO OLEFIN UNIT

(71) Applicant: UOP LLC, Des Plaines, IL (US)

(72) Inventors: Andrea G. Bozzano, Northbrook, IL (US); Steven L. Krupa, Fox River Grove, IL (US); Timothy Foley, Forest Park, IL (US)

(73) Assignee: UOP LLC, Des Plaines, IL (US)

(21) Appl. No.: 13/852,047

(22) Filed: Mar. 28, 2013

(51) Int. Cl. C07C 5/327; C07C 1/22

(52) U.S. Cl. CPC C07C 5/327; C07C 1/22
USPC 585/254; 585/324; 585/327; 585/329

(57) ABSTRACT

A method of producing butene from an oxygenate-containing feedstock is described. The oxygenate-containing feedstock is converted to olefins and separated. The C₄ isoolefins are then etherified and separated. The normal C₄ olefins can be used to produce butadiene.

### BACKGROUND OF THE INVENTION

Currently, butadiene comes from steam cracking of petroleum feedstocks. In the steam cracking of hydrocarbons such as ethane, liquefied petroleum gas, naphtha, and gasoil, a steam cracking product is produced which comprises olefins such as ethylene, propylene, butylene, and heavier hydrocarbons. The composition of the heavier hydrocarbons from the stream cracking process will vary according to the feedstock charged to the steam cracking reaction zone. The lighter the feedstock, the more light olefins are produced. As the steam cracking feedstock increases in carbon number, the more aromatics are formed among the heavier hydrocarbons. Generally, the C₄ fraction, produced by the steam cracking reaction may contain as much as 45 weight percent di-olefins as butadiene, and about 50 to about 60 weight percent mono-olefins such as normal butenes and iso-butenes. Approximately 15 to about 25 weight percent of the C₄ fraction comprises iso-butylene. The steam cracking process is well known to those of ordinary skill in the art. Steam cracking processes are generally carried out in radiant furnace reactors at elevated temperatures for short residence times while maintaining a low reactant partial pressure, relatively high mass velocity, and effecting a low pressure drop through the reaction zone.

However, it is expected that future production of butadiene from steam crackers will fall short of demand because the feedstocks to steam crackers are becoming lighter, with a shift away from naphtha feed to ethane feed. Consequently, there will be a need for the intentional production of butadiene. One problem which this raises is where an appropriate feed source for the production of butadiene can be found. Such a feed would desirably contain normal butenes, with little or no isobutene. Typically, this feed would come from steam cracking. However, it is expected that there will be a shortage of butenes from steam crackers for the same reason the butadiene shortage is expected, the shift to lighter feeds to the steam cracker.

Therefore, there is a need for an economical and substantial feedstock of normal butenes with little or no isobutene.

### SUMMARY OF THE INVENTION

One aspect of the invention involves a method of producing butene from an oxygenate-containing feedstock. In one embodiment, the method comprises contacting the oxygenate-containing feedstock in an oxygenate conversion reactor with an oxygenate conversion catalyst at reaction conditions effective to convert the oxygenate-containing feedstock to an oxygenate conversion effluent stream comprising light olefins and C₄₊ hydrocarbons, wherein the light olefins comprise ethylene and propylene and the C₄₊ hydrocarbons comprise butenes and pentenes, the butenes comprising n-butene and isobutenes, and the pentenes comprising n-pentene and isopentenes. The oxygenate conversion effluent stream is separated in a separation zone into a light olefin stream and a C₄₋ hydrocarbon stream. The C₄₊ hydrocarbon stream is contacted with an etherification catalyst in an etherification reaction zone at etherification conditions to react the isobutenes and tertiary isopentenes with an alcohol to produce an etherification effluent stream comprising n-butenes, n-pentenes, and ethers, the ethers comprising methyl tert-butyl ether and tert-amyl methyl ether. The etherification effluent stream is separated into an ether stream and an olefin stream comprising n-butenes and n-pentenes.

Another aspect of the invention involves a method of producing butadiene from an oxygenate-containing feedstock. In one embodiment, the method comprises contacting the oxygenate-containing feedstock in an oxygenate conversion reactor with an oxygenate conversion catalyst at reaction conditions effective to convert the oxygenate-containing feedstock to an oxygenate conversion effluent stream comprising light olefins and C₄ hydrocarbons, wherein the light olefins comprise ethylene and propylene and the C₄₊ hydrocarbons comprise butenes and pentenes, the butenes comprising n-butene and isobutenes, and the pentenes comprising n-pentene and isopentenes. The oxygenate conversion effluent stream is separated in a separation zone into a light olefin stream and a C₄₊ hydrocarbon stream. The C₄₊ hydrocarbon stream is contacted with an etherification catalyst in an etherification reaction zone at etherification conditions to react the isobutenes and tertiary isopentenes with an alcohol to produce an etherification effluent stream comprising n-butene, n-pentene, and ethers, the ethers comprising methyl tert-butyl ether and tert-amyl methyl ether. The etherification effluent stream is separated into an ether stream and an olefin stream comprising n-butene and n-pentene. The olefin stream is separated into an n-butene stream and an n-pentene stream. The n-butene stream is contacted with a dehydrogenation catalyst in a dehydrogenation reaction zone under dehydrogenation conditions to form the butadiene.

### BRIEF DESCRIPTION OF THE DRAWING

FIG. 1 is an illustration of one embodiment of the process of the present invention.

FIG. 2 is an illustration of an alternative embodiment of the process of the present invention.

### DETAILED DESCRIPTION OF THE INVENTION

The invention solves the problem of the shortage of feedstock for butadiene production by processing the C₄ and C₅ olefins produced in oxygenate to olefins (OTO) processes, e.g., a methanol to olefin (MTO) process, to co-produce normal butenes and pentenes along with methyl tert-butyl ether (MTBE) and tert-amyl methyl ether (TAME). The normal butenes can be used to produce butadiene.

The etherification of the combined C₄ and C₅ olefin stream improves the economics of the process. The C₄ olefin stream alone is too small due to the small amount of isobutene produced. However, the inclusion of the C₅ olefins improves the overall yield of ether.

OTO processes, in particular the MTO process, are today being used for conversion of alcohols, such as methanol, to light olefins, namely ethylene and propylene. These processes are highly selective to production of ethylene and propylene, but in some cases also have some byproduct production of C₄₊ olefins. In conventional processing, the C₄₋ olefin byproduct from an OTO unit can be sent to an Olefin Cracking Process Unit, or OCP, in which the olefins are further cracked to produce an additional amount of light olefins.

It has been found that with the proper catalyst choice, the C₄ olefin byproduct from an OTO unit is high in concentration of normal butene, and low in concentration of paraffins and branched olefins. Hence, the C₄ olefin byproduct from OTO is a highly suitable feedstock for production of normal butenes, and for production of butadiene. However, the C₄ olefin stream contains some small concentration of isobutene. It is important to note that even low concentrations of isobutene can be problematic for downstream processing. For example, isobutene co-boils with 1-butene, and must be removed prior to 1-butene recovery. Also, it is known that isobutene can be problematic in the oxidative dehydrogenation of butene to butadiene, since isobutene can lead to formation of unwanted byproducts. Hence, even though dilute, isobutene must be removed from the C₄ olefin stream. This step can be carried out through ethers formation.

It has also been found that the C₅ olefin byproduct from an OTO unit is considerably more highly branched, with significant amounts of isopentene. It is well known that isopentene can be reacted with methanol to produce tert-amyl methyl ether (TAME).

This invention seeks to utilize the synergy of the need for removal of isobutene with the opportunity for production of MTBE and TAME through an ethers unit. The invention also seeks to make use of the synergy of a common oxygenate feed being used for both feeding the OTO unit and the ethers production unit.

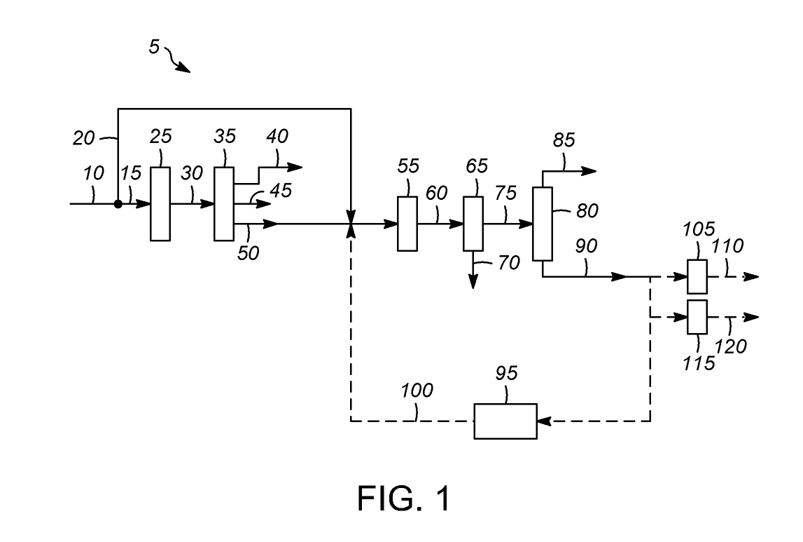

A simplified process 5 is illustrated in FIG. 1. A feed 10 of methanol, for example, is divided into two portions 15, 20. Feed 15 is sent to the MTO reaction zone 25 for conversion to olefins. The effluent 30 contains a mixture of C₂, C₃, C₄, and C₅ olefins, with minimal amounts of C₆ olefins. The effluent 30 is separated in a separation zone 35 into a C₂ stream 40, a C₃ stream 45, and a C₄₊ stream 50. A C₄₊ stream includes butenes, pentenes, and higher olefins.

As used herein, the term “zone” can refer to an area including one or more equipment items and/or one or more sub-zones. Equipment items can include one or more reactors or reactor vessels, heaters, exchangers, pipes, pumps, compressors, and controllers. Additionally, an equipment item, such as a reactor, dryer, or vessel, can further include one or more zones or sub-zones.

The C₄ stream from the MTO process is highly linear, with about 2% isobutene and about 1% butadiene. The C₅ stream from the MTO unit has slightly more branching, with a concentration of isopentenes of about 25% or greater. The combined C₄ and C₅ stream 50 and the second portion of the methanol feed 20 is fed to an ether production unit 55 in which the isobutene is converted to MTBE, and the tertiary isopentenes(2-methyl-1-butene and 2-methyl-2-butene) are converted to TAME. The effluent 60 from the ether production unit 55 is sent to a separation zone 65 where it is separated into an ether stream 70 and an olefin stream 75. The olefin stream 75 from the ether production unit 55 is reasonably free of iso-olefins.

The olefin stream 75 is routed to a distillation column 80 where it is separated into a normal butene stream 85 and a normal pentene stream 90.

The normal butene stream 85 can optionally be separated into 1-butene and 2-butene for recovery of the 1-butene, and the 2-butene can be sent for dehydrogenation to butadiene. Alternatively, the mixture of 1-butene and 2-butene can be dehydrogenated to butadiene. The dehydrogenation of butenes to butadiene can be carried out through conventional catalytic dehydrogenation routes or through oxidative dehydrogenation routes.

The normal pentene stream 90 can be sent for further processing. In one case, the normal pentenes can be isomerized in an isomerization unit 95 to isopentene, and the isopentene stream 100 recycled to the ether production unit 55 to produce additional TAME. In some cases, it may be more desirable to send the pentene isomerization effluent to a separate TAME reaction system (not shown). In another embodiment, the normal pentene stream 95 can be saturated in a hydrogenation unit 105 to produce a C₅ paraffin stream 110 for use as a possible blending component for gasoline. If hydrogen is produced from the dehydrogenation of butene to butadiene, it can be used for the saturation. Another option is to use the normal pentene stream 95 as a dimerization or oligomerization feedstock in an oligomerization unit 115 to produce a C₁₀₊ product stream 120. Such a product would be useful as a distillate stream, or perhaps a reformer feed to make an aromatics rich C₁₀ stream.

The first step is the MTO process, more generally the oxygenate conversion process, in which an oxygenate feedstock is catalytically converted to hydrocarbons containing aliphatic moieties, including, but not limited to, methane, ethane, ethylene, propane, propylene, butylene, and limited amounts of other higher aliphatics such as pentenes, by contacting the oxygenate feedstock with a preselected catalyst. The oxygenate feedstock comprises hydrocarbons containing aliphatic moieties, including, but not limited to, alcohols, halides, mercaptans, sulfides, amines, ethers, carbonyl compounds, or mixtures thereof. The aliphatic moiety preferably contains from about 1 to about 10 carbon atoms, and more preferably 1 to about 4 carbon atoms. Representative oxygenates, include, but are not limited to, methanol, isopropanol, n-propanol, ethanol, fuel alcohols, dimethyl ether, diethyl ether, methyl mercaptan, methyl sulfide, methyl amine, ethyl mercaptan, ethyl chloride, formaldehyde, dimethylketone, acetic acid, n-alkylamines, n-alkylhalides, and n-alkyl-sulfides having alkyl groups of 1 to 10 carbon atoms, or mixtures thereof. In one embodiment, methanol is used as the oxygenate feedstock.

A diluent can be used to maintain the selectivity of the oxygenate conversion catalyst to produce light olefins, particularly ethylene and propylene. Steam is commonly used as the diluent.

The oxygenate conversion process can be conducted in the vapor phase such that the oxygenate feedstock is contacted in a vapor phase in a reaction zone with a non-zeolite molecular sieve catalyst at effective process conditions to produce hydrocarbons, i.e., an effective temperature, pressure, WHSV and, optionally, an effective amount of diluent, correlated to produce olefins having 2 to 4 carbon atoms per molecule, with smaller amounts of higher olefins, such as pentenes. The olefins produced by the oxygenate conversion zone include ethylene, propylene, butylenes, and pentenes. In general, the residence time employed to produce the desired olefin product can vary from seconds to a number of hours. It will be appreciated that the residence time will be determined to a significant extent by the reaction temperature, the molecular sieve selected, the WHSV, the phase (liquid or vapor), and the process design characteristics selected. The oxygenate feedstock flow rate affects olefin production.

Suitable conditions for the oxygenate conversion process are well known. Pressures range from 0.1 kPa (0.001 atm) to about 101 MPa (1000 atm), or about 1.0 kPa (0.01 atm) to about 10.1 MPa (100 atm), or about 101 kPa (1 atm) to about 1.01 MPa (10 atm). The pressures referred to herein for the oxygenate conversion process are exclusive of the inert diluent, if any, that is present and refer to the partial pressure of the feedstock as it relates to oxygenate compounds and/or mixtures thereof. The temperature which may be employed in the oxygenate conversion process may vary over a wide range depending, at least in part, on the molecular sieve catalyst used. In general, the process can be conducted at an effective temperature between about 200° C. (392° F.) and about 700° C. (1292° F.). The reaction can occur at pressures and temperatures outside these ranges, although perhaps not as well as within the ranges.

The selection of a particular catalyst for use in the oxygenate conversion process depends upon the particular oxygenate conversion process and other factors known to those skilled in the art which need not be further discussed herein. The catalysts desirably have relatively small pores. The preferred small pore catalysts are defined as having pores at least a portion, desirably a major portion, of which have an average effective diameter characterized such that the adsorption capacity (as measured by the standard McBain-Bakr gravimetric adsorption method using given adsorbate molecules) shows adsorption of oxygen (average kinetic diameter of about 0.346 nm) and negligible adsorption of isobutane (average kinetic diameter of about 0.5 nm). Certain of the catalysts useful in the present invention have pores with an average effective diameter of less than 5 Angstroms. The average effective diameter of the pores of the catalysts is determined by measurements described in D. W. Breck, Zeolite Molecular Sieves, John Wiley & Sons, New York (1974), hereby incorporated by reference in its entirety. The term effective diameter is used to denote that occasionally the pores are irregularly shaped, e.g., elliptical, and thus the pore dimensions are characterized by the molecules that can be adsorbed rather than the actual dimensions. Desirably, the small pore catalysts have a substantially uniform pore structure, e.g., substantially uniformly sized and shaped pore. Suitable catalysts can be chosen from among layered clays, zeolitic molecular sieves, and non-zeolitic molecular sieves.

Zeolitic molecular sieves in the calcined form can be represented by the general formula:

Me_{2/n}O:Al₂O₃:xSiO₂:yH₂O

where Me is a cation, x has a value from about 2 to infinity, n is the cation valence and y has a value of from about 2 to 10.

Typically, well-known zeolites which may be used include chabazite (also referred to as Zeolite D), clinoptilolite, erionite, faujasite (also referred to as Zeolite X and Zeolite Y), ferrierite, mordenite, Zeolite A, Zeolite P, ZSM-5, ZSM-11, and MCM-22. Other zeolites include those having a high silica content, i.e., those having silica to alumina ratios greater than 10 and typically greater than 100, can also be used. One such high silica zeolite is silicalite; as the term is used herein, it includes both the silicapolymorph disclosed in U.S. Pat. No. 4,061,724, and also the F-silicate disclosed in U.S. Pat. No. 4,073,865, hereby incorporated by reference.

Non-zeolitic molecular sieves include molecular sieves which have the proper effective pore size and are embraced by an empirical chemical composition, on an anhydrous basis, expressed by the empirical formula:

(ElₓAl_{y}P_{z})O₂

where EL is a metal selected from the group consisting of silicon, magnesium, zinc, iron, cobalt, nickel, manganese, chromium and mixtures thereof, x is the mole fraction of EL and is at least 0.005, y is the mole fraction of Al and is at least 0.01, z is the mole fraction of P and is at least 0.01 and x+y+z=1. When EL is a mixture of metals, x represents the total amount of the metal mixture present. Preferred metals (EL) are silicon, magnesium and cobalt, with silicon being especially preferred.

The catalyst for the oxygenate conversion zone can be incorporated into solid particles in which the catalyst is present in an amount effective to promote the desired hydrocarbon conversion. In one aspect, the solid particles comprise a catalytically effective amount of the catalyst and at least one matrix material, preferably selected from the group consisting of binder materials, filler materials, and mixtures thereof to provide a desired property or properties, e.g., desired catalyst dilution, mechanical strength, and the like to the solid particles. Such matrix materials are often to some extent porous in nature and may or may not be effective to promote the desired hydrocarbon conversion. The matrix materials may promote conversion of the feedstream and often provide reduced selectivity to the desired product or products relative to the catalyst. Filler and binder materials include, for example, synthetic and naturally occurring substances such as metal oxides, clays, silicas, alms, silica-aluminas, silica-magnesias, silica-zirconias, silica-thorias, silica-berylias, silica-titanias, silica-alumina-thorias, silica-alumina-zirconias, aluminophosphates, mixtures of these and the like. If matrix materials, e.g., binder and/or filler materials, are included in the catalyst composition, the non-zeolitic and/or zeolitic molecular sieves preferably comprise about 1% to 99%, more preferably about 5% to about 90% and still more preferably about 10% to about 80%, by weight of the total composition. The preparation of solid particles comprising catalyst and matrix materials is conventional and well known in the art and, therefore, need not be discussed in detail herein.

The etherification step of the C₄₋ stream produces MTBE from iso-butylene and methanol and TAME by reacting the tertiary C₅ iso-olefins with methanol. Etherification reactions are carried out in the presence of an acid catalyst such as a sulfonated, macroporous organic ion exchange resin in the liquid phase at temperatures between about 30 and about 100° C.

The alcohol will enter the etherification zone along with the alkene reactants. Contained in the etherification zone is an etherification catalyst which, upon contact with the alcohol and isoalkene and normal alkene hydrocarbons, will produce the ether product. A wide range of materials are known to be effective as etherification catalysts for the isoalkene reactants including mineral acids such as sulfuric acid, boron trifluoride, phosphoric acid on kieselguhr, phosphorous-modified zeolites, heteropoly acids, and various sulfonated resins. The use of a sulfonated solid resin catalyst is preferred. These resin type catalysts include the reaction products of phenolformaldehyde resins and sulfuric acid and sulfonated polystyrene resins including those crosslinked with divinylbenzene. A particularly preferred etherification catalyst is a macroporous acid-form of a sulfonic ion exchange resin such as a sulfonated styrene-divinylbenzene resin as described in U.S. Pat. No. 2,922,822 having a degree of crosslinking of about 5 to 60%. Suitable resins are available commercially. Specialized resins have been described in the art and include copolymers of sulfonyl fluorovinyl ether and fluorocarbons as described in U.S. Pat. No. 3,489,243. Another specially prepared resin consists of the SiO₂-modified cation exchangers described in U.S. Pat. No. 4,751,343. The macroporous structure of a suitable resin is described in detail in U.S. Pat. No. 5,012,031 as having a surface area of at least about 400 m²/g, a pore volume of about 0.6-2.5 ml/g and a mean pore diameter of 40-1000 Angstroms. It is contemplated that the subject process could be performed using a metal-containing resin which contains one or more metals from sub-groups VI, VII or VIII of the Periodic Table such as chromium, tungsten, palladium, nickel, chromium, platinum, or iron as described in U.S. Pat. No. 4,330,679. Further information on suitable etherification catalysts may be obtained by reference to U.S. Pat. Nos. 2,480,940, 2,922,822, and 4,270,929.

A wide range of operating conditions can be employed in processes for producing ethers from olefins and alcohols. Many of these include vapor, liquid, or mixed-phase operations. Processes operating with vapor or mixed-phase conditions may be suitably employed in this invention. In a preferred embodiment, liquid phase conditions are used.

The range of etherification conditions for processes operating in liquid phase includes a broad range of suitable conditions including a superatmospheric pressure sufficient to maintain the reactants as liquid phase, generally below about 4.8 MPa(g) (700 psig), and a temperature between about 29.4° C. (85° F.) and about 98.9° C. (210° F.). Even in the presence of additional light materials, pressures in the range of about 0.97 MPa(g) (140 psig) to 4.0 MPa(g)(580 psig) are sufficient. A preferred temperature range is about 37.8° C. (100° F.) to about 98.9° C. (210° F.). The reaction rate is normally faster at higher temperatures, but conversion is more complete at lower temperatures due to more favorable thermodynamic equilibrium. High conversion can, therefore, be obtained by splitting the reaction zone into multiple stages, possibly with inter-cooling between reactor stages or with the use of an isothermal tubular reactor, so that the final reactor stage can operate at the lower temperature as desired to reach the highest equilibrium conversion of tertiary iso-olefins. This may be accomplished most easily with two reactors. The ratio of alcohol to isoolefin should normally be maintained in the range of about 1:1 to 2:1, preferably 1.05:1 and 1.5:1. A description of suitable etherification processes useful for the present invention can be found in U.S. Pat. Nos. 4,219,678 to Obenaus et aL., and U.S. Pat. No. 4,282,389 to Droste et aL., which are incorporated herein.

The etherification zone operates selectively to convert principally only the tertiary olefins. Therefore, the normal alkenes pass through the etherification zone with minimal conversion to products or by-products. Reactor conditions are typically optimized so that undesired n-olefin reaction products, such as methyl sec-butyl ether are minimized in the ether product. Thus, the etherification zone effluent provides a stream of ether product and normal alkenes for separation.

The effluent from the etherification reaction exits the etherification reaction zone and enters a separation zone. The separation zone can be any zone known to those skilled in the art for separating a hydrocarbon feed stream into its various fractions. In a preferred embodiment, the arrangement of the separation zone typically consists of at least one distillation zone. A number of distillation arrangements may be possible to separate the unreacted methanol, the unreacted C4 and C5 alkenes, and the product ethers. As a possible fractionation scheme, a first column can be used to separate unreacted alcohol and unreacted n-butene in the overhead from TAME, MTBE, and unreacted pentene in the bottoms. The bottoms product can then be routed to a next column, in which n-pentene is recovered in the overhead and TAME/MTBE are recovered in the bottoms.

A useful arrangement for the separation zone of this invention is the use of reactive distillation columns containing one or more beds of etherification catalyst. The distillation zone can provide additional etherification of unreacted isobutene and tertiary isopentenes. Accordingly, the reactive distillation zone can be used as a combined reactor. Processes for the production of ethers by reactive distillation are taught in U.S. Pat. Nos. 3,634,535 and 4,950,803. The operating conditions employed in the reactive distillation zone are generally the same as those outlined herein for the etherification reaction zone. No particular apparatus or arrangement is needed to retain the catalyst bed within the distillation section of the reactive distillation zone and a variety of methods can be used to incorporate the bed or region of catalyst within the reactive distillation zone. For example, the catalyst may be retained between suitable packing materials or may be incorporated onto a distillation tray itself. A preferred method of retaining the catalyst is through the use of a corrugated structural device that is described in U.S. Pat. No. 5,073,236 which is hereby incorporated by reference.

The fractionation scheme using reactive distillation columns is similar to the one described above. The reactor product can enter a first reactive distillation column, in which unreacted isobutene is converted to MTBE. The overhead product from this column would consist of unreacted methanol and n-butene, while the bottoms could consist of unreacted pentene, MTBE and TAME. The bottoms would be routed to a second column, optionally a reactive distillation column, in which additional isopentene would be reacted to TAME, and the unconverted n-pentene would be recovered in the overhead, while the product MTBE and TAME would be recovered in the bottoms.

It is also possible, through careful design and choice of operating conditions, to accomplish both conversion of isobutene and conversion of isopentene in a single reactive distillation column in some cases, depending on product specifications.

The unconverted n-pentene is also suitable for processing in different ways. One option is to route the n-pentene to an olefin skeletal isomerization reaction section. Olefin skeletal isomerization is a practiced technology for the conversion of normal olefins to iso-olefins. This type of technology utilizes vapor phase reaction conditions and produces equilibrium mixtures of olefins. A commercial example of this technology is the Trans4m Technology offered by Lyondell Bassel. The effluent from the skeletal isomerization section can now be suitably routed to an etherification reaction zone, either the first etherification reaction zone, or a separate, dedicated etherification reaction zone.

An alternate processing route for the unconverted n-pentene is to route it to a dimerization or oligomerization section. In this section, the n-pentene can be converted to decene or greater. Decene produced is suitable for feedstock to a reformer. Higher carbon number oligomers can be suitable for use in the distillate pool.

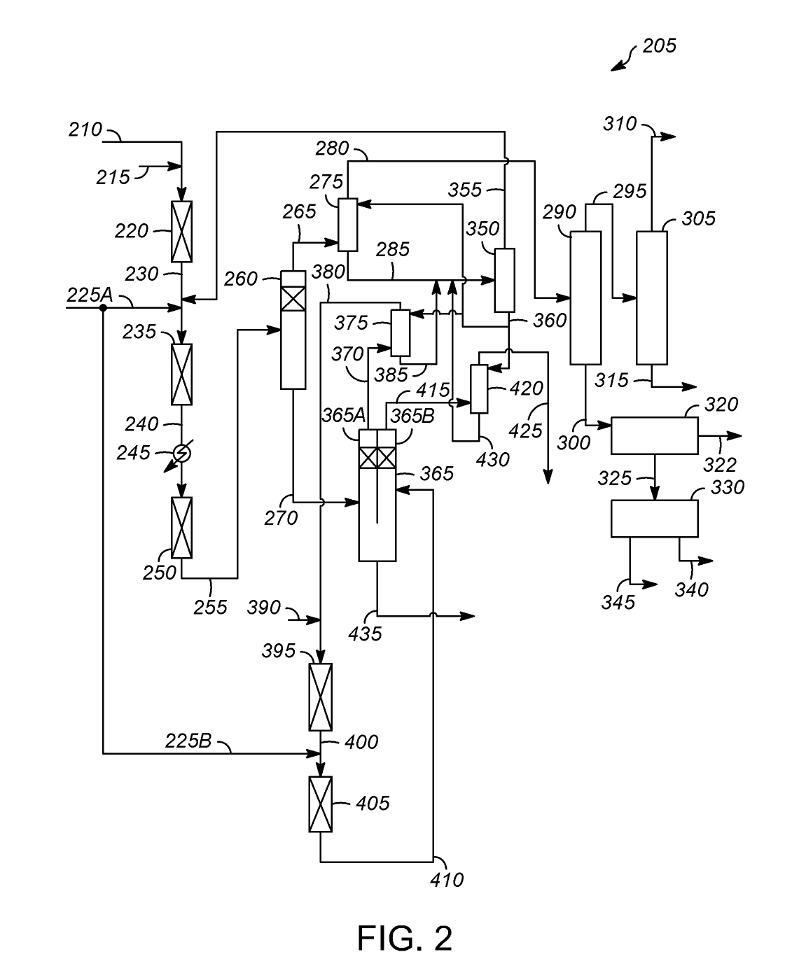

FIG. 2 illustrates one embodiment of process 205 including an etherification process with butene and pentene separation. The C₄₊ stream 210 from the MTO process is mixed with hydrogen 215 and sent to an optional selective hydrogenation reaction zone 220 where any dienes present are reacted with the hydrogen 215. This reaction is desirable because isoprene and other C₅ dienes will potentially be reactive in the ether unit and lead to color bodies in the TAME product. There is also a potential for gum formation due to C₁₀ diene type products that fractionate with the TAME. In addition, if it is desired to include butene-1 recovery as part of the flow scheme, this reaction will hydrogenate any 1,3-butadiene in the feed coming from the MTO process. The product specifications for butadiene in butene-1 are very low, about 30 wppm, so even trace ppm butadiene in the fresh feed must be removed by hydrogenation to butenes because isobutene and 1,3-butadiene cannot be separated by fractionation.

Methanol 225A is mixed with the effluent 230 from the selective hydrogenation reaction zone 220 and sent to the first ether reaction zone 235. The effluent 240 from the first etherification zone 235 is cooled in a heat exchanger 245 and sent to the second etherification zone 250. The effluent 255 from the second etherification zone 250 is sent to a reactive distillation column 260 where the effluent 255 is separated into an overhead stream 265 comprising butenes and methanol and a bottoms stream 270 comprising pentenes, MTBE, and TAME.

The overhead stream 265 is sent to a first water washing zone 275 where it is separated into a stream 280 comprising butenes and a stream 285 comprising the methanol and water. The stream 280 comprising the butenes is sent to a first separation zone 290 where it is separated into an overhead stream 295 comprising C₃₋, dimethyl ether (DME), butene-1, and isobutene, and a bottoms stream 300 comprising butene-2 and normal butane.

The overhead stream 295 is sent to a second separation zone 305 where it is separated into an overhead stream 310 comprising C₃₋ and DME and a bottoms stream 315 comprising butene-1. The reactive distillation zone 260 is designed to meet (i.e., isobutene conversion level) whatever product specification is desired in stream 315 with respect to the maximum acceptable isobutene content.

The bottoms stream 300 from the first separation zone 290 is sent to a dehydrogenation zone 320 where the butene-2 is dehydrogenated to form 1,3-butadiene. The dehydrogenation produces hydrogen stream 322. The effluent 325 from the dehydrogenation zone 320 is sent to an extraction zone 330 where it is separated into a stream 340 comprising C₄ raffinate, and a stream 345 comprising 1,3-butadiene.

The stream 285 comprising the methanol and water from the first water washing zone 275 is sent to separation zone 350 where it is separated into an overhead stream 355 comprising methanol and a bottoms stream 360 comprising water. The overhead stream 355 comprising methanol is recycled back and mixed with the effluent 230 from the selective hydrogenation reaction zone 220.

The bottoms stream 270 comprising pentenes, MTBE, and TAME from the reactive distillation column 260 is sent to a second reactive distillation column 365. This is desirably a divided wall column to avoid the cost of alternately using two separate reactive distillation columns and to minimize isopentene losses in the net C5 product stream 425.

The bottoms stream 270 is sent to one side 365A of the second reactive distillation column 365. The overhead stream 370 from the first side 365A comprising pentenes and methanol is sent to a second water washing zone 375 where it is separated into a stream 380 comprising isopentene and normal pentene and a stream 385 comprising methanol and water. The stream 385 is mixed with stream 285 and sent to separation zone 350 to be separated into methanol and water.

The stream 380 comprising isopentene and normal pentene is mixed with hydrogen 390 and sent to an isomerization reaction zone 395 where the normal pentene is isomerized. The effluent 400 from the isomerization reaction zone 395 is mixed with methanol 225B and sent to a third etherification zone 405. The effluent 410 from the third etherification zone 405 is sent to the second side 365B of the second reactive distillation column 365.

The overhead stream 415 from the second side 365B comprising normal pentene depleted in tertiary isopentenes is sent to a third water washing zone 420 where the stream 425 comprising normal pentene depleted in tertiary isopentenes is separated from a stream 430 comprising water and methanol. The stream 430 is mixed with streams 285 and 385 and sent to the separation zone 350 where the water and methanol are separated.

The bottoms stream 360 from the separation zone 350 is sent to the first, second, and third water washing zones, 275, 375, and 420.

The stream 425 comprising normal pentene can be processed as described above in FIG. 1, as desired.

The bottoms stream 435 from the reactive distillation column 365 comprising MTBE and TAME can be recovered.

Although FIG. 2 shows removing methanol from the column overhead streams using water washing followed by a methanol column, other approaches can also be used. Suitable approaches include, but are not limited to, adsorbent based systems.

It will be appreciated by one skilled in the art that various features of the above described process, such as pumps, instrumentation, heat-exchange and recovery units, condensers, compressors, flash drums, feed tanks, and other ancillary or miscellaneous process equipment that are traditionally used in commercial embodiments of hydrocarbon conversion processes have not been described or illustrated. It will be understood that such accompanying equipment may be utilized in commercial embodiments of the flow schemes as described herein. Such ancillary or miscellaneous process equipment can be obtained and designed by one skilled in the art without undue experimentation.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A method of producing butene from an oxygenate-containing feedstock comprising:
contacting the oxygenate-containing feedstock in an oxygenate conversion reaction zone with an oxygenate conversion catalyst at reaction conditions effective to convert the oxygenate-containing feedstock to an oxygenate conversion effluent stream comprising light olefins and C₄₊ hydrocarbons, wherein the light olefins comprise ethylene and propylene and the C₄₊ hydrocarbons comprise butenes and pentenes, the butenes comprising n-butene and isobutenes, and the pentenes comprising n-pentene and isopentenes;
separating the oxygenate conversion effluent stream in a separation zone into a light olefin stream and a C₄₊ hydrocarbon stream;
contacting the C₄₊ hydrocarbon stream with an etherification catalyst in an etherification reaction zone at etherification conditions to react the isobutenes and tertiary isopentenes with an alcohol to produce an etherification effluent stream comprising n-butenes, n-pentenes, and ethers, the ethers comprising methyl tert-butyl ether and tert-amyl methyl ether;
separating the etherification effluent stream into an ether stream and an olefin stream comprising n-butene and n-pentene.

2. The method of claim 1 further comprising separating the olefin stream into an n-butene stream and an n-pentene stream.

3. The method of claim 2 further comprising contacting the n-butene stream with a dehydrogenation catalyst in a dehydrogenation reaction zone under dehydrogenation conditions to form butadiene.

4. The method of claim 2 further comprising:
contacting the n-pentene stream with an isomerization catalyst in an isomerization reaction zone under isomerization conditions to produce an isomerized isopentene stream comprising isopentenes and n-pentene; and
routing the isomerized isopentene stream to the etherification reaction zone.

5. The method of claim 2 further comprising recovering the n-butene stream.

6. The method of claim 2 further comprising recovering the n-pentene stream.

7. The method of claim 2 further comprising contacting the n-pentene stream with a hydrogenation catalyst in a hydrogenation reaction zone under hydrogenation conditions to form an n-pentane stream.

8. The method of claim 2 further comprising oligomerizing the n-pentene stream to produce a C₁₀₊ distillate stream.

9. The method of claim 1 wherein the oxygenate-containing feedstock comprises C₁ C₅ monohydroxy alcohol.

10. The method of claim 1 wherein the alcohol comprises a C₁ to C₅ monohydroxy alcohol.

11. The method of claim 1 wherein the oxygenate-containing feedstock comprises methanol.

12. The method of claim 1 wherein the alcohol comprises methanol.

13. A method of producing butadiene from an oxygenate-containing feedstock comprising:
contacting the oxygenate-containing feedstock in an oxygenate conversion reaction zone with an oxygenate conversion catalyst at reaction conditions effective to convert the oxygenate-containing feedstock to an oxygenate conversion effluent stream comprising light olefins and C₄₊ hydrocarbons, wherein the light olefins comprise ethylene and propylene and the C₄₊ hydrocarbons comprise butenes and pentenes, the butenes comprising n-butene and isobutenes, and the pentenes comprising n-pentene and isopentenes;
separating the oxygenate conversion effluent stream in a separation zone into a light olefin stream and a C₄₊ hydrocarbon stream;
contacting the C₄₊ hydrocarbon stream with an etherification catalyst in an etherification reaction zone at etherification conditions to react the isobutenes and tertiary isopentenes with an alcohol to produce an etherification effluent stream comprising n-butene, n-pentene, and ethers, the ethers comprising methyl tert-butyl ether and tert-amyl methyl ether;
separating the etherification effluent stream into an ether stream and an olefin stream comprising n-butene and n-pentene;
separating the olefin stream into an n-butene stream and an n-pentene stream;
contacting the n-butene stream with a dehydrogenation catalyst in a dehydrogenation reaction zone under dehydrogenation conditions to form the butadiene.

14. The method of claim 13 further comprising:
contacting the n-pentene stream with an isomerization catalyst in an isomerization reaction zone under isomerization conditions to produce an isomerized isopentene stream comprising isopentenes and n-pentene; and
routing the isomerized isopentene stream to the etherification reaction zone.

15. The method of claim 13 further comprising recovering the n-pentene stream.

16. The method of claim 13 further comprising contacting the n-pentene stream with a hydrogenation catalyst in a hydrogenation reaction zone under hydrogenation conditions to form an n-pentane stream.

17. The method of claim 13 further comprising oligomerizing the n-pentene stream to produce a C₁₀₊ distillate stream.

18. The method of claim 13 wherein the oxygenate-containing feedstock comprises C₁ to C₅ monohydroxy alcohol.

19. The method of claim 13 wherein the alcohol comprises a C₁ to C₅ monohydroxy alcohol.

20. The method of claim 1 wherein the oxygenate-containing feedstock comprises methanol and wherein the alcohol comprises methanol.
